(19) United States
(12) Patent Application Publication
(10) Pub. No.: US 2008/0015358 A1
(43) Pub. Date: Jan. 17, 2008

(54) Fluorination Process of Protected Aminothiazole

(76) Inventors: Matthew Colin Thor Fyfe, Oxfordshire (GB); Frederic Naud, Basel (CH)

Correspondence Address: ELI LILLY & COMPANY, PATENT DIVISION, P.O. BOX 6288, INDIANAPOLIS, IN, 46206-6288, US

(21) Appl. No.: 11/573,582

(22) PCT Filed: Aug. 12, 2005
(86) PCT No.: PCT/GB05/03170

§ 371 Date: Feb. 12, 2007

(30) Foreign Application Priority Data
Aug. 12, 2004 (GB) 04118058.4

(51) Int. Cl. C07D 277/40; C07D 277/38

(52) U.S. Cl. 548/195; 548/199

(57) ABSTRACT

A process for the production of fluorinated compound formula (I) comprising fluorination of a protected aminothiazole. Compounds formula (I) are useful in the preparation of activators of glucokinase.

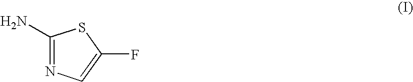

### BACKGROUND OF THE INVENTION

The present invention is directed to a process for the production of fluorinated compounds. In particular, the invention is directed to a process for the production of a fluorinated compound of use in the production of pharmaceutically active compounds, especially compounds which are useful as activators of glucokinase for the treatment of type II diabetes.

International Patent Applications PCT/US04/03968 and PCT/GB2005/050053 (published after the priority date of the present application) disclose various tri(cyclo) substituted amide compounds which are modulators of glucokinase and are useful in the prophylactic or therapeutic treatment of hyperglycemia and type II diabetes. Certain of these compounds, for example (2R)-2-(4-cyclobutanesulfonylphenyl)-N-(5-fluorothiazol-2-yl)-3-(tetrahydropyran-4-yl)propionamide, (2R)-2-(4-cyclopropanesulfonylphenyl)-N-(5-fluorothiazol-2-yl)-3-(tetrahydropyran-4-yl)propionamide and 2(R)-2-(4-cyclopropanesulfonylphenyl)-N-(5-fluorothiazol-2-yl)-3-((R)-3-oxocyclopentyl)propionamide, contain a 5-fluorothiazole group. There is a need for efficient processes for the production of 2-amino-5-fluorothiazole and acid addition salts thereof, e.g. the hydrochloride salt, which are useful as intermediates for the synthesis of the therapeutic compounds.

2-Amino-5-fluorothiazole is disclosed by name in U.S. Pat. No. 4,094,785, U.S. Pat. No. 4,086,240, DE2724614 and U.S. Pat. No. 4,046,768, however no methods for the synthesis of this compound are disclosed. The production of 2-amino-5-fluorothiazole trifluoroacetate by addition of trifluoroacetic acid to a solution of (5-fluorothiazol-2-yl)carbamic acid tert-butyl ester is described in WO2004/063179 but no details for the preparation of the carbamic acid ester starting material or characterization of the product are provided. PCT/US04/03968 describes the synthesis of 2-amino-5-fluorothiazole hydrochloride from 5-bromothiazol-2-ylamine hydrobromide via N-(5-bromothiazol-2-yl)-2,2,2-trifluoroacetamide. However, this process is not particularly efficient for the synthesis of such compounds on a commercial scale. Therefore, there is a need for further efficient processes for the production of 2-amino-5-fluorothiazole.

### SUMMARY OF THE INVENTION

A process for the production of 2-amino-5-fluorothiazole or an acid addition salt thereof.

### DETAILED DESCRIPTION OF THE INVENTION

The present invention provides a process for the production of a compound of formula (I): 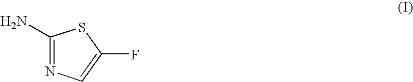
or an acid addition salt thereof, comprising fluorination of a compound of formula (II): 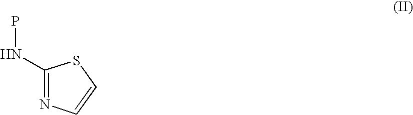
wherein P is a protecting group, followed by removal of the protecting group and optional salt formation.

Protecting groups that P may represent include any amino protecting groups such as those described in Protective Groups in Organic Chemistry, T. W. Greene and P. G. M. Wuts, (1991) Wiley-Interscience, New York, 2^{nd} edition. Particular protecting groups which may be mentioned include acetyl, pivaloyl and tert-butoxycarbonyl(Boc), a preferred protecting group is tert-butoxycarbonyl.

In a first and preferred embodiment of the invention the fluorination reagent used in the method is an electrophilic fluorinating agent e.g. comprising an active N-fluorine bond. Examples of electrophilic fluorinating agents include N-fluorosulfonamides and N-fluorosulfonimides as described for example in A. J. Poss et al., Speciality Chemicals Magazine, April 2003, 36-40 and E. C. Taylor et al., Org. Prep. Proceed. Int., 1997, 29, 221-223. Preferred fluorinating reagents are N-fluorosulfonimides, a particularly preferred fluorinating agent is N-fluorobenzenesulfonimide.

The fluorination is preferably conducted at reduced temperature, for example a temperature of about −50° C.

The dianion of the compound of formula (II) is preferably prepared prior to addition of the fluorination reagent by deprotonation with an appropriate base e.g. an organolithium or organomagnesium reagent e.g. a Grignard reagent. Preferred bases are organolithium reagents e.g. n-, tert-, or sec-butyl lithium, methyl lithium and phenyl lithium, a particularly preferred base is tert-butyl lithium. Preferably at least 2 equivalents, especially about 2 equivalents e.g. 2.2 equivalents, of the base relative to the compound of formula (II) are used.

The dianion of the compound of formula (II) is stable for several hours at a temperature of e.g. from about −50 to 0° C.

In this preferred embodiment the fluorination reaction is preferably conducted in a suitable solvent, preferably a non-polar aprotic solvent such as ether, tetrahydrofuran or dioxane, preferably tetrahydrofuran.

In a second embodiment the reagent is an electrophilic aromatic substitution reagent such as 1-chloromethyl-4-fluoro-1,4-diazoniabicyclo[2.2.2]octane bis(tetrafluoroborate) (Selectfluor®), see G. S. Lal, J. Org. Chem., 1993, 58, 2791-2796.

In this second embodiment the fluorination reaction is preferably conducted in a suitable solvent, for example acetonitrile.

In this second embodiment the fluorination reaction is preferably conducted at an elevated temperature, for example the reflux temperature of the solvent.

Prior to removal of the protecting group the fluorinated intermediate produced from the compound of formula (I) according to the method of the invention may be further purified by recrystallisation. A suitable recrystallisation solvent is a mixture of trifluoroethanol and formic acid, e.g. at a ratio of about 100:1 v/v.

Suitable acid addition salts of 2-amino-5-fluorothiazole include those formed with inorganic and organic acids. Such acids include, for example, acetic, trifluoroacetic, benzenesulfonic, benzoic, camphorsulfonic, citric, ethanesulfonic, fumaric, gluconic, glutamic, hydrobromic, hydrochloric, hydrofluoric isethionic, lactic, maleic, malic, mandelic, methanesulfonic, mucic, nitric, pamoic, pantothenic, phosphoric, succinic, sulfuric, tartaric, p-toluenesulfonic, triflic acid and the like. Particularly preferred are the hydrohalide salts especially the hydrochloride.

Acid addition salts of 2-amino-5-fluorothiazole may be prepared by reaction of the amine with the appropriate acid. The hydrochloride salt is preferably prepared by dissolving the amine in a suitable solvent e.g. tetrahydrofuran or dioxane, preferably dioxane, and bubbling through HCl gas. The resulting hydrochloride salt may be isolated by the addition of a cosolvent, e.g. diethylether, and filtration of the resulting solid.

The compounds of formula (II) may be prepared from 2-aminothiazole by methods known to those skilled in the art, for example as described by C. Poupat, Tetrahedron, 58, 2002, 4201-4215.

The invention also provides the use of the compounds of formula (I) prepared as described above as an intermediate for the manufacture of a compound of formula (III), or a pharmaceutically acceptable salt thereof:

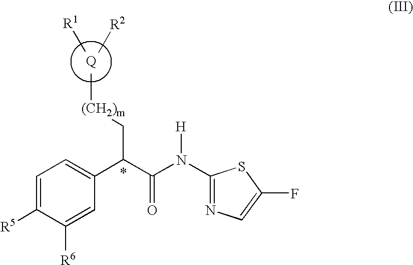

wherein Q is an aryl, a 5- or 6-membered heteroaryl, or a 4-8-membered heterocyclic ring;

R¹ and R² each independently are hydrogen, hydroxy, halogen, cyano, nitro, vinyl, ethynyl, methoxy, OCFₙH₃₋ₙ, —N(C₀₋₄alkyl)(C₀₋₄alkyl), CHO, or C₁₋₂alkyl optionally substituted with 1-5 independent halogen, hydroxy, cyano, methoxy, —N(C₀₋₂alkyl)(C₀₋₂alkyl), SOCH₃, or SO₂CH₃ substituents; or R¹ and R² together form a carbocyclic or heterocyclic ring; or R¹ and R² may be taken together to represent an oxygen atom attached to the ring via a double bond;

R⁵ and R⁶ each independently are hydrogen, hydroxy, halogen, cyano, nitro, CO₂R⁷, CHO, COR⁸, C(OH)R⁷R⁸, C(═NOR⁷)R⁸, CONR⁹R¹⁰, SR⁷, SOR⁸, SO₂R⁸, SO₂NR⁹R¹⁰, CH₂NR⁹R¹⁰, NR⁹R¹⁰, N(C₀₋₄alkyl)SO₂R⁸, NHCOR⁷, or a C₁₋₄alkyl group, C₂₋₄alkenyl group, C₂₋₄alkynyl group, C₁₋₄alkoxy group, aryl group, or heteroaryl group, wherein any group optionally is substituted with 1-6 independent halogen, cyano, nitro, hydroxy, C₁₋₂alkoxy, —N(C₀₋₂alkyl)(C₀₋₂alkyl), C₁₋₂alkyl, CFₙH₃₋ₙ, aryl, heteroaryl, —COC₁₋₂alkyl, —CON(C₀₋₂alkyl)(C₀₋₂alkyl), SCH₃, SOCH₃, SO₂CH₃, or —SO₂N(C₀₋₂alkyl)(C₀₋₂alkyl) substituents; or R⁵ and R⁶ together form a 5-8-membered carbocyclic or heterocyclic ring;

R⁷ is hydrogen, or a C₁₋₄alkyl group, C₂₋₄alkenyl group, C₂₋₄alkynyl group, C₃₋₇cycloalkyl group, aryl group, heteroaryl group, or 4-7-membered heterocyclic group, wherein any group optionally is substituted with 1-6 independent halogen, cyano, nitro, hydroxy, C₁₋₂alkoxy, —N(C₀₋₂alkyl)(C₀₋₂alkyl), C₁₋₂alkyl, C₃₋₇cycloalkyl, 4-7-membered heterocyclic ring, CFₙH₃₋ₙ, aryl, heteroaryl, CO₂H, —COC₁₋₂alkyl, —CON(C₀₋₂alkyl)(C₀₋₂alkyl), SOCH₃, SO₂CH₃, or —SO₂N(C₀₋₂alkyl)(C₀₋₂alkyl) substituents;

R⁸ is a C₁₋₄alkyl group, C₂₋₄alkenyl group, C₂₋₄alkynyl group, C₃₋₇cycloalkyl group, aryl group, heteroaryl group, or 4-7-membered heterocyclic group, wherein any group optionally is substituted with 1-6 independent halogen, cyano, nitro, hydroxy, C₁₋₂alkoxy, —N(C₀₋₂alkyl)(C₀₋₂alkyl), C₁₋₂alkyl, C₃₋₇cycloalkyl, 4-7-membered heterocyclic ring, CFₙH₃₋ₙ, aryl, heteroaryl, CO₂H, COC₁₋₂alkyl, —CON(C₀₋₂alkyl)(C₀₋₂alkyl), SOCH₃, SO₂CH₃, or —SO₂N(C₀₋₂alkyl)(C₀₋₂alkyl) substituents;

R⁹ and R¹⁰ each independently are hydrogen, or a C₁₋₄alkyl group, C₃₋₇cycloalkyl group, aryl group, heteroaryl group, or 4-7-membered heterocyclic group, wherein any group optionally is substituted with 1-6 independent halogen, cyano, nitro, hydroxy, C₁₋₂alkoxy, —N(C₀₋₂alkyl)(C₀₋₂alkyl), C₁₋₂alkyl, C₃₋₇cycloalkyl, 4-7-membered heterocyclic ring, CFₙH₃₋ₙ, aryl, heteroalkyl, COC₁₋₂alkyl, —CON(C₀₋₂alkyl)(C₀₋₂alkyl), SOCH₃, SO₂CH₃, or —SO₂N(C₀₋₂alkyl)(C₀₋₂alkyl) substituents; or R⁹ and R¹⁰ together form a 6-8-membered heterobicyclic ring system or a 4-8-membered heterocyclic ring which optionally is substituted with 1-2 independent C₁₋₂alkyl, CH₂OCH₃, COC₀₋₂alkyl, hydroxy, or SO₂CH₃ substituents;

n is 1, 2 or 3; and

m is 0 or 1.

In the compounds of formula (III) the carbon atom linking the aryl ring and Q-bearing sidechain to the carbonyl carbon is a chiral centre. Accordingly, the compound may be present either as a racemate, or as a single enantiomer in the (R)- or (S)-configuration. The (R)-enantiomers are preferred.

The compounds of formula (III) may be prepared by the condensation of the amine of formula (I) or a salt thereof, with a carboxylic acid of formula (IV):

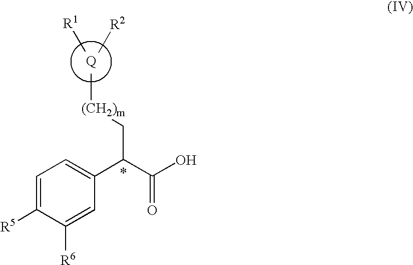

wherein R¹, R², R⁵, R⁶, Q and m are as defined for formula (III), using a variety of coupling conditions, e.g. polymer supported carbodiimide-1-hydroxybenzotriazole in N,N-dimethylformamide at 20° C. (for representative procedures, see http://www.argotech.com/PDF/resins/ps_carbodiimide.pdf and available from Argonaut Technologies, Inc., Foster City, Calif.). Preferably the condensation is performed employing a reagent that minimises racemisation of the chiral centre, e.g. benzotriazol-1-yloxytris(pyrrolidino)phosphonium hexafluorophosphate (J. Coste et al. Tetrahedron Lett. 1990, 31, 205-208), to furnish enantiopure (R)-amides of Formula (III).

Alternatively the coupling reaction may employ an activated derivative of the carboxylic acid of formula (IV), for example a protected ester or acid chloride thereof which may be prepared by methods known to those skilled in the art, in which case the coupling may be conducted in the presence of collidine or another suitable pyridine derivative.

The carboxylic acids of formula (IV) may be prepared by reaction of a compound of formula (V) with a compound of formula (VI):

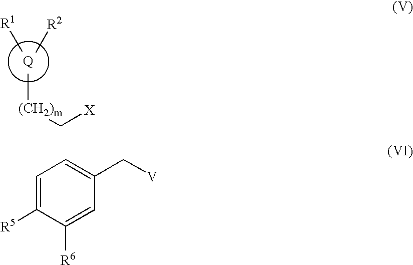

wherein R¹, R², R⁵, R⁶, Q and m are as defined above, V is CO₂R¹¹ or CO₂CH₂Ph, and X is chloro, bromo, iodo, or —OSO₂R¹²; wherein R¹¹ is C₀₋₄alkyl and R¹² is C₁₋₄alkyl, optionally substituted with one or more fluorines, or optionally substituted aryl.

The halides and sulfonate esters (V) are commercially available or are readily prepared using known techniques. These alkylating agents may be reacted with the dianions of the phenylacetic acids (VI), generated at −78° C. in tetrahydrofuran with ≧2 equivalents of a strong base, such as lithium diisopropylamide, to generate (IV) directly (F. T. Bizzarro et al., WO 00/58293). Alternatively, the α-carbanion of phenylacetic ester (VI), generated at −78° C. in tetrahydrofuran by a strong base, such as lithium bis(trimethylsilyl)amide (L. Snyder et al., J. Org. Chem. 1994, 59, 7033-7037), can be alkylated by (V) to give α-substituted esters. Saponification of these esters, employing, for example, sodium hydroxide in aqueous methanol at 20° C. to reflux, leads to the carboxylic acids (IV).

The carboxylic acids of formula (IV) may alternatively be synthesized by enantioselective hydrogenation of the corresponding (E)-2-(4-cycloalkanesulfonylphenyl)-3-(tetrahydropyran-4-yl)acrylic acid as described in the Examples.

Preferred compounds of formula (III) prepared according to this aspect of the invention include those compounds in which:

Q is preferably 2-furyl, 2-thienyl, tetrahydropyranyl, tetrahydrothiopyranyl, 1-oxo-tetrahydrothiopyranyl, or 1,1-dioxo-tetrahydrothiopyranyl; more preferably 4-tetrahydropyranyl or 4-tetrahydrothiopyranyl; most preferably 4-tetrahydropyranyl.

When Q is a heteroaryl or heterocyclic group it is preferably linked to the —(CH₂)ₘ-group through a carbon atom.

When Q is a heteroalkyl group it preferably does not have a substituent R¹ or R² other than hydrogen at a position adjacent to point of attachment to the —(CH₂)ₘ— group.

R¹ and R² are preferably hydrogen.

R⁵ and R⁶ are preferably not both hydrogen.

R⁵ is preferably CF₃, SOR⁸, SO₂R⁸, SO₂NR⁹R¹⁰, NHSO₂R⁸, or triazolyl; more preferably SOR⁸, SO₂R⁸, or SO₂NR⁹R¹⁰; most preferably SO₂R⁸ or SO₂NR⁹R¹⁰, especially SO₂R⁸. In particular R⁵ is SO₂C₃₋₄cycloalkyl, especially SO₂cyclopropyl.

R⁶ is preferably hydrogen, chloro, fluoro, or trifluoromethyl; more preferably hydrogen.

R⁷ and R⁸ are preferably C₁₋₄alkyl, C₃₋₇cycloalkyl, heteroaryl, or 4-7-membered heterocyclic group; more preferably C₁₋₃alkyl, 4-6-membered heterocyclic group, or C₃₋₅cycloalkyl; most preferably methyl, ethyl, n-propyl, cyclopropyl, cyclobutyl, oxetanyl, or tetrahydrofuryl, and especially methyl, ethyl, n-propyl, cyclopropyl, or cyclobutyl, especially cyclopropyl.

When R⁵ and/or R⁶ are CO₂R⁷ or SR⁷, R⁷ is preferably not hydrogen.

R⁹ and R¹⁰ are preferably independently C₀₋₄alkyl e.g. one of R⁹ and R¹⁰ is hydrogen and the other is ethyl, or combine to form a 4-8-membered heterocyclic ring. R⁹ and R¹⁰ are preferably not both hydrogen.

m is preferably 0.

n is preferably 2 or 3.

A preferred group of compounds are compounds of Formula (III), or pharmaceutically acceptable salts thereof, wherein:

Q is 4-tetrahydropyranyl;

R¹ and R² are hydrogen;

R⁵ is SO₂R⁸, or SO₂NR⁹R¹⁰;

R⁶ is hydrogen;

R⁸ is a C₃₋₅cycloalkyl group or a 4-6-membered heterocyclic group, and, in addition;

R⁹ and R¹⁰ are independently C₀₋₄alkyl, provided that R⁹ and R¹⁰ are not both hydrogen; and

m is 0.

A more preferred group of compounds are compounds of Formula (III), or pharmaceutically acceptable salts thereof, wherein:

Q is 4-tetrahydropyranyl;

R¹ and R² are hydrogen;

R⁵ is SO₂R⁸;

R⁶ is hydrogen;

R⁸ is a C₃₋₅cycloalkyl group; and

m is 0.

The invention also provides the use of the compounds of formula (I) prepared as described above as an intermediate for the manufacture of a compound of formula (VII), or a pharmaceutically acceptable salt thereof:

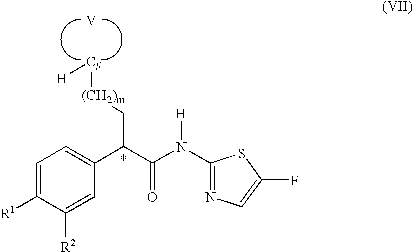

wherein V is (CH₂)ₖ where one CH₂ group may optionally be replaced by CH(OH), C═O, C═NOH, C═NOCH₃, CHX, CXX¹, CH(OCH₃), CH(OCOCH₃), CH(C₁₋₄alkyl), or C(OH)(C₁₋₄alkyl);

X and X¹ are independently selected from fluoro and chloro;

R¹ and R² are independently selected from hydrogen, halogen, hydroxy, amino, cyano, nitro, SR³, SOR³, SO₂R³, SO₂NR⁴R⁵, NHSO₂R³, or a C₁₋₄alkyl, C₂₋₄alkenyl, C₂₋₄alkynyl, C₁₋₄alkoxy, or heteroaryl group, wherein any group is optionally substituted with 1 to 5 substituents independently selected from halogen, cyano, nitro, hydroxy, C₁₋₂alkoxy, —N(C₀₋₂alkyl)(C₀₋₂alkyl), C₁₋₂alkyl, CFₙH₃₋ₙ, aryl, heteroaryl, —CON(C₀₋₂alkyl)(C₀₋₂alkyl), SCH₃, SOCH₃, SO₂CH₃, and —SO₂N(C₀₋₂alkyl)(C₀₋₂alkyl);

R³ is a C₁₋₄alkyl group, C₃₋₇cycloalkyl group, aryl group, heteroaryl group, or 4- to 7-membered heterocyclic group, wherein any group is optionally substituted with 1 to 5 substituents independently selected from halogen, cyano, nitro, hydroxy, C₁₋₂alkoxy, —N(C₀₋₂alkyl)(C₀₋₂alkyl), C₁₋₂alkyl, C₃₋₇cycloalkyl, 4- to 7-membered heterocyclic ring, CFₙH₃₋ₙ, aryl, heteroaryl, COC₁₋₂alkyl, —CON(C₀₋₂alkyl)(C₀₋₂alkyl), SOCH₃, SO₂CH₃, and —SO₂N(C₀₋₂alkyl)(C₀₋₂alkyl);

R⁴ and R⁵ are independently hydrogen, or a C₁₋₄alkyl group, C₃₋₇cycloalkyl group, aryl group, heteroaryl group, or 4- to 7-membered heterocyclic group, wherein any group is optionally substituted with 1 to 5 substituents independently selected from halogen, cyano, nitro, hydroxy, C₁₋₂alkoxy, —N(C₀₋₂alkyl)(C₀₋₂alkyl), C₁₋₂alkyl, C₃₋₇cycloalkyl, 4- to 7-membered heterocyclic ring, CFₙH₃₋ₙ, aryl, heteroaryl, —CON(C₀₋₂alkyl)(C₀₋₂alkyl), SOCH₃, SO₂CH₃, and —SO₂N(C₀₋₂alkyl)(C₀₋₂alkyl);

or R⁴ and R⁵ together form a 4- to 8-membered heterocyclic ring which is optionally substituted with 1 or 2 substituents independently selected from C₁₋₂alkyl and hydroxy;

k is an integer from 2 to 7;

m is 0 or 1; and

n is 1, 2 or 3.

In the compounds of formula (VII) the carbon atom linking the aryl ring and the —HC< >V-bearing sidechain to the carbonyl carbon is a chiral centre. Accordingly, the compound may be present either as a racemate, or as a single enantiomer in the (R)- or (S)-configuration. The (R)-enantiomers are preferred.

The compounds of formula (VII) may be prepared by the condensation of the amine of formula (I) or a salt thereof, with a carboxylic acid of formula (VIII) or an activated derivative thereof:

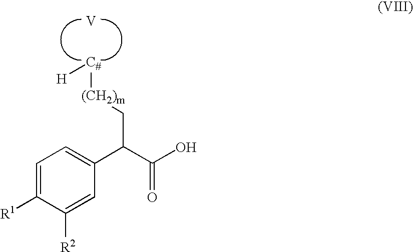

wherein V, R¹, R² and m are as defined for formula (VII) using a variety of coupling conditions as described above for the synthesis of the compounds of formula (III).

The carboxylic acids of formula (VIII) may be prepared by reaction of a compound of formula (IX) with a compound of formula (X):

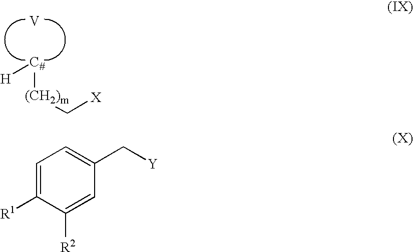

wherein V, R¹, R² and m are as described above, Y is CO₂R¹² wherein R¹² is hydrogen, C₁₋₄alkyl or benzyl; and X is chloro, bromo, iodo, or —OSO₂R¹³, wherein R¹³ is C₁₋₄alkyl, optionally substituted with one or more fluorines, or optionally substituted aryl.

The halides and sulfonate esters (IX) and the phenylacetic acids and esters (X) are commercially available or are readily prepared using known techniques, for example as described in International Patent Publication Nos. WO2000/058293, WO2001/044216 and WO2003/095438. These alkylating agents may be reacted with the dianions of the phenylacetic acids (X), generated at −78° C. in tetrahydrofuran with ≧2 equivalents of a strong base, such as lithium diisopropylamide, to generate (VII) directly (F. T. Bizzarro et al., WO2000/58293). Alternatively, the α-carbanion of phenylacetic ester (X), generated at −78° C. in tetrahydrofuran by a strong base, such as lithium bis(trimethylsilyl)amide (L. Snyder et al., J. Org. Chem. 1994, 59, 7033-7037), can be alkylated by (IX) to give α-substituted esters. Saponification of these esters, employing, for example, sodium hydroxide in aqueous methanol at 20° C. to reflux, leads to the carboxylic acids (VII).

Preferred compounds of formula (VII) prepared according to this aspect of the invention include those compounds in which:

The group formed by —HC< and >V represents oxocycloalkyl or hydroxycycloalkyl, e.g. 3-oxocyclopentyl particularly (R)-3-oxocyclopentyl, 4-oxocyclohexyl or 3-hydroxycyclopentyl, especially (R)-3-oxocyclopentyl.

R¹ and R² are not both hydrogen.

R¹ is CF₃, SOR³, SO₂R³, SO₂NR⁴R⁵, NHSO₂R³, or triazolyl; more preferably SOR³, SO₂R³, or SO₂NR⁴R⁵; most preferably SO₂R³ or SO₂NR⁴R⁵, especially SO₂R³. In particular R¹ is SO₂C₃₋₄cycloalkyl, especially SO₂cyclopropyl.

R² is hydrogen, chloro, fluoro, or trifluoromethyl; more preferably hydrogen or chloro.

R³ is C₁₋₃alkyl or C₃₋₄cycloalkyl, more preferably C₃₋₄cycloalkyl, especially cyclopropyl.

R⁴ and R⁵ are independently hydrogen or C₁₋₄alkyl, e.g. one of R⁴ and R⁵ is hydrogen and the other is ethyl, or combine to form a 4- to 8-membered heterocyclic ring. R⁴ and R⁵ are preferably not both hydrogen.

m is 0.

V is (CH₂)ₖ where one CH₂ group is replaced by CH(OH) or C═O.

k is 4 or 5.

Various functional groups present in the compounds described above and intermediates for use in the preparation thereof may be produced by functional group conversions known to those skilled in the art. For example sulfonyl groups may be produced by oxidation of the corresponding sulfanyl group using e.g. mCPBA.

Further details for the preparation of the compounds are found in the examples.

During the synthesis of the compounds described above, labile functional groups in the intermediate compounds, e.g. hydroxy, oxo, carboxy and amino groups, may be protected. The protecting groups may be removed at any stage in the synthesis of the compounds. A comprehensive discussion of the ways in which various labile functional groups may be protected and methods for cleaving the resulting protected derivatives is given in, for example, Protective Groups in Organic Chemistry, T. W. Greene and P. G. M. Wuts, (1991) Wiley-Interscience, New York, 2^{nd} edition.

The invention also provides a pharmaceutical composition comprising a compound of formula (III) or (VII), or a pharmaceutically acceptable salt thereof, produced according to the method described above, in combination with a pharmaceutically acceptable diluent or carrier.

The invention also provides a method of prophylactic or therapeutic treatment of a condition where activation of glucokinase is desirable comprising a step of administering an effective amount of a compound of formula (III) or (VII), produced according to the method described above, or a pharmaceutically acceptable salt thereof.

The invention also provides a method of prophylactic or therapeutic treatment of hyperglycemia or diabetes, particularly type II diabetes, comprising a step of administering an effective amount of a compound of formula (III) or (VII), produced according to the method described above, or a pharmaceutically acceptable salt thereof. In this aspect of the invention the compound of formula (III) or (VII), may be administered in combination with one or more other anti-hyperglycemic agents or anti-diabetic agents.

The invention also provides a method of prevention of diabetes, particularly type II diabetes, in a human demonstrating pre-diabetic hyperglycemia or impaired glucose tolerance comprising a step of administering an effective prophylactic amount of a compound of formula (III) or (VII), produced according to the method described above, or a pharmaceutically acceptable salt thereof.

All publications, including, but not limited to, patents and patent application cited in this specification, are herein incorporated by reference as if each individual publication were specifically and individually indicated to be incorporated by reference herein as fully set forth.

The invention will now be described by reference to the following examples which are for illustrative purposes and are not to be construed as a limitation of the scope of the present invention.

### EXAMPLES

### Materials and Methods

Column chromatography was carried out on SiO₂ (40-63 mesh). LCMS data were obtained using a Waters Symmetry 3.5μ C₁₈ column (2.1×30.0 mm, flow rate 0.8 mL/min) eluting with solvent A (5% MeCN in H₂O) and solvent B (MeCN solution containing 0.1% HCO₂H) over 6 min and UV detection at 220 nm. Gradient information: 0.0-1.2 min: 100% A; 1.2-3.8 min: Ramp up to 10% A-90% B; 3.8-4.4 min: Hold at 10% A-90% B; 4.4-5.5 min: Ramp up to 100% B; 5.5-6.0 min: Return to 100% A. The mass spectra were obtained employing an electrospray ionisation source in the positive (ES⁺) ion mode. Prep HPLC purification was carried out using a Lunar 10μ ODS2 (250×21.2 mm; flow rate 20 mL/min) eluting with solvent A (0.05% TFA, 10% MeCN, 90% water) and solvent B (0.05% TFA, 90% MeCN, 10% water) and UV detection at 215 nm. Gradient information: 0.0-0.2 min: 90% A, 10% B; 0.2-10.0 min: Ramp up to 10% A, 90% B; 10.0-15.0 min: 10% A, 90% B; 15.0-16.0 min: Return to 90% A, 10% B.

### Preparation 1: Ethyl (4-cyclopropylsulfanylphenyl)oxoacetate

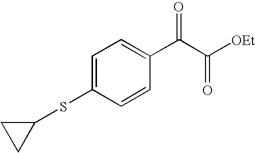

AlCl₃ (104.6 g, 0.79 mol) was suspended in CH₂Cl₂ (1.15L) and cooled in an ice/salt bath to 0° C. with stirring. Ethyl chlorooxoacetate (84.8 g, 0.62 mol) was then added over a period of 10 min, during which time the temperature was maintained between 0 and 2° C. The mixture was then stirred for a further 30 min at 0° C., before the addition of cyclopropylphenylsulfide (85.0 g, 0.57 mol) over a period of 45 min, during which time the temperature remained between 0 and 8° C. The resulting mixture was allowed to warm to room temperature and stirred for a further 2 h. After this time ice/water (275 mL) was added, with ice bath cooling maintaining the temperature at 20° C. The organic layer was separated and washed with water (2×250 mL), saturated NaHCO₃ solution (2×250 mL) and again with water (1×250 mL). The organic fraction was then dried (MgSO₄) filtered and the solvent removed to provide the title compound (134 g, 94% yield). NMR was consistent with the above structure.

### Preparation 2: Ethyl (4-cyclopropylsulfonylphenyl)oxoacetate

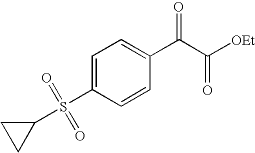

To a stirred solution of Preparation 1 (49.4 g, 0.2 mol) in CH₂Cl₂ (180 mL) was added a solution of m-chloroperoxybenzoic acid (92.0 g, 0.40 mol, calc as 75% strength) in CH₂Cl₂ (650 mL) over 45 min with the temperature maintained at 15-25° C. TLC(CH₂Cl₂:ethyl acetate 1:10) showed that starting material still remained. Further m-chloroperoxybenzoic acid (3.4 g) in CH₂Cl₂ was added and the reaction stirred for 30 min. A second TLC still showed the presence of some starting material, and additional m-chloroperoxybenzoic acid (3.4 g) was added and the reaction stirred for a further 2 h. TLC showed a small amount of starting material so a final quantity of m-chloroperoxybenzoic acid (1.0 g) was added and the reaction continued for 1 h. Sodium carbonate solution (2M, 500 mL) was then added and the aqueous layer was separated, the pH raised to 9-10 and reextracted with CH₂Cl₂. The organic extracts were combined, washed with water (2×400 ml), dried (MgSO₄), filtered and the solvent removed under vacuum (54.1 g, 96% yield). NMR was consistent with the above structure.

### Preparation 3: (Tetrahydropyran-4-yl)methanol

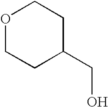

To a suspension of LiAlH₄ (56 g, 1.47 mol) in diethyl ether (2L) under argon was added methyl tetrahydro-2H-pyran-4-carboxylate (270 g, 1.88 mol) in diethyl ether (ca. 200 mL) under reflux over a period of 1.75 h. After addition was complete reflux was continued for a further 1 h. TLC (diethyl ether) indicated a small amount of ester remained, so further LiAlH₄ (10 g, 0.26 mol) was added and reflux continued for 1 h. Water (66 mL) was added, then 15% NaOH solution (66 mL), followed by further water (198 mL). The solid was filtered and dried to give the crude product, which was redissolved in DCM (800 ml), dried (MgSO₄), filtered and the solvent removed to afford the title compound (207 g, 94% yield). NMR was consistent with the above structure.

### Preparation 4: Methanesulfonicacid (tetrahydropyran-4-yl)methyl ester

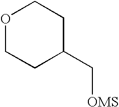

To a mixture of Preparation 3 (216.5 g, 1.87 mol) and triethylamine (299 mL) in DCM (1.3L) at <10° C. was added under argon a solution of methanesulfonyl chloride (236 g, 160 mL) in DCM (200 mL) over 2 h 50 min, maintaining the temperature at 5-10° C. throughout. Subsequent washing with water (1L), 1M HCl (500 mL), 5% NaHCO₃ (300 mL), water (300 mL), drying (MgSO₄) and then removal of the solvent afforded the title compound (328 g, 90% yield). NMR was consistent with the above structure.

### Preparation 5: 4-Iodomethyltetrahydropyran

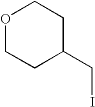

A mixture of Preparation 4 (328 g, 1.69 mol) and sodium iodide (507 g, 3.4 mol) in acetone (3.3L) was refluxed for 4 h. TLC (diethyl ether) showed significant mesylate remaining so further sodium iodide (127 g, 0.65 mol) was added and reflux continued for 16 h. The mixture was cooled and filtered. The resulting cake was washed with acetone, dried, and then partitioned between diethyl ether (800 mL) and water (800 mL). The aqueous phase was re-extracted with diethyl ether (200 mL), the ether extracts combined and washed with 10% sodium thiosulphate solution (300 mL) which decolourised the extract. Final washing with water (300 mL), drying (MgSO₄) and then removal of the solvent provided the title compound (365 g, 92% yield). NMR was consistent with the above structure.

### Preparation 6: Triphenyl(tetrahydropyran4-ylmethyl)phosphonium iodide

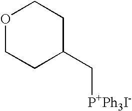

A mixture of Preparation 5 (350 g, 1.55M) and triphenylphosphine (406 g, 1.55M) in acetonitrile (1.6L) was heated under reflux. After 27 h the mixture was cooled and filtered, washed with diethyl ether and dried in air to provide a white solid (504 g). Filtrate and washings were returned to reflux and concentrated to 750 mL, reflux was maintained for 16 h before cooling and dilution with diethyl ether (ca 1.2L). A precipitate formed which was stirred for 30 min before being filtered, washed with diethyl ether (2×300 mL) and dried in air to yield a further crop (10 g). Overall yield of the title compound (604 g, 80%). RT=2.7 min; m/z (ES⁺)=361.2.

### Preparation 7: (E)-2-(4-Cyclopropanesulfonylphenyl)-3-(tetrahydropyran-4-yl)acrylic acid

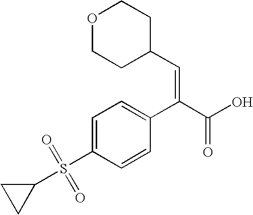

To a suspension of Preparation 6 (2.49 kg, 5.10 mol) in dry THF (5L) maintained between −5 and 0° C. was added a solution of lithium hexamethyldisilazide (1.05M, 4.39 kg, 5.18 mol) over 30 min. The resulting mixture was then warmed to 15° C. and stirred for 2 h before recooling to between 0 and 5° C. A solution of Preparation 2 (1.25 kg, 4.43 mmol) in THF (2.5L) was then added over 1 h, during which time the temperature was maintained between 0 and 5° C., before a period of 16 h at between 20 and 25° C. Subsequently, brine (17% w/w, 3.8L) was added and the phases separated with the aid of additional brine (1.3L). The aqueous phase was reextracted with methyl t-butyl ether (2×2.5L) and the combined organic extracts washed with brine (2×3.8L). The solvents were removed under vacuum at between 30 and 40° C. The residue was dissolved in methanol (15L) and aqueous sodium hydroxide (2M, 4.34L) added before heating at 65-67° C. for 4 h. The mixture was cooled and the solvents removed under vacuum at between 35 and 40° C. until water started to distil. The residue was diluted with water (15L). The solid phosphine oxide was filtered off, washed with water (2.5L) and the filtrate separated. The aqueous phase was washed with methyl t-butyl ether (5L and 3.5L), before acidification with hydrochloric acid solution (5M, 1.9L) in the presence of methyl t-butyl ether (10L). The organic phase was separated and the aqueous phase reextracted with methyl t-butyl ether (5L). The combined organic extracts were washed with saturated brine (2×1L) and the solvent removed under vacuum. Methanol (2L) was added and then removed under vacuum, this step was then repeated. The residue was brought to a total weight of 4.0 kg by addition of methanol and stirred at ambient temperature to crystallise the product. Filtration of the solid and washing with chilled (ca 0° C.) methanol (500 mL) gave, after vacuum drying at 40° C., the title compound (654 g, 41% yield after correction for residual solvent). NMR was consistent with the above structure.

### Preparation 8: (2R)-2-(4-cyclopropanesulfonylphenyl)-3-(tetrahydropyran-4-yl)propionic acid

(E)-2-(4-Cyclopropanesulfonylphenyl)-3-(tetrahydropyran-4-yl)acrylic acid (Preparation 7, 110 g, 0.327 mol) was dissolved in MeOH/Toluene 5:1 (1.4L). In a 40 mL Schlenk flask was placed [Rh(nbd)₂](BF₄) (30.5 mg, 0.08 mmol) and All-MOD-Mandyphos (90.4 mg, 0.08 mmol), dissolved in MeOH (10 mL) and stirred for 1 h at RT. This catalyst solution was then added to the (E)-2-(4-cyclopropanesulfonylphenyl)-3-(tetrahydropyran-4-yl)acrylic acid solution and transferred to a 2.5L autoclave. The autoclave was pressurized to 50 bar and heated to 30° C. After 18 h the pressure was released and the solution transferred to a 3L flask. Active charcoal (3 g) was added to the reaction mixture, stirred for 1 h and the charcoal removed by filtration. The solution was further filtered over Hyflo and a Zeta-Bond filter. The solution thus obtained was concentrated under partial pressure and the solid obtained further dried under high vacuum to give a solid (105 g). The solid was placed in a 1.5L flask equipped with a mechanical stirrer, a thermometer and a dropping funnel. Isobutylacetate (540 mL) was added at RT and the suspension heated at 110° C. until a clear solution was observed. Heptane (60 mL) was added slowly at 110° C., the oil bath was then removed and the solution allowed to cool slowly. The reaction was stirred for a further 16 h, the title compound filtered off and dried under high vacuum (77.2 g, 70% yield, 99% ee). ¹H NMR (CDCl₃, 300.13 MHz) δ: 7.85 (2H, Aryl H, d, J_{HH}=6.6 Hz), 7.50 (2H, Aryl H, d, J_{HH}=6.6 Hz), 3.95 (br d, 2H), 3.80 (t, 1H, CHCH₂, J_{HH}=7.8 Hz), 3.35 (m, 2H), 2.45 (m, 1H), 2.10 (m, 1H), 1.75 (m, 1H), 1.60 (m, 2H), 1.50-1.20 (m, 5H), 1.05 (m, 2H).

### Example 1

### a) 2-(Tert-butoxycarbonylamino)-5-fluorothiazole

2-(Tert-butoxycarbonylamino)thiazole (10 g, 0.050 mol) in THF (0.2L) was cooled to −50° C. under argon. tBuLi solution in pentane (60 mL of a 1.7M solution, 0.102 mol, 2.05 eq) was added over a period of 30 nm in and the temperature kept below −40° C. The suspension thus obtained was stirred at −50° C. for 30 min. A solution of N-fluorobenzenesulfonimide (NFSi) was prepared (22.0 g, 0.07 mol in 70 mL THF, 1.4 eq) and 50 mL of this solution (1 eq) was added over a 5 min period and the temperature kept under −40° C. The reaction was stirred for 20 min at −50° C. Then tBuLi (10 mL, 0.017 mol, 0.35 eq) and the NFSi solution (10 mL, 0.4 eq) added. The solution thus obtained was stirred at −50° C. for 45 nm and then added to saturated NH₄Cl solution (300 mL). The organic phase was separated and the aqueous phase further washed with diethylether (100 mL). The combined organic fractions were washed with brine (20 mL) solution and dried (Na₂SO₄). The solvent was removed and the solid further dried to afford a brown solid. To this crude product was added trifluoroethanol (60 mL) and formic acid (0.6 mL). The suspension was heated to 85° C. until it gave a solution. The flask was then cooled to RT and the precipitate thus formed filtered off to afford, after drying under high vacuum, the title compound (6.4 g, contains 2.3% of starting material according to HPLC at 275 nm). After a second crystallisation (trifluoroethanol (22 mL) and formic acid (0.22 mL) for 20 min at 85° C.), the title compound was obtained as an off white solid (4.6 g, contains 1% of starting material, 97.5% pure by HPLC). ¹H NMR (CDCl₃) δ: 6.90 (1H, d, CHCF), 1.60 (9H, s, Boc-H).

### b) 5-Fluorothiazol-2-ylamine hydrochloride

2-(Tert-butoxycarbonylamino)-5-fluorothiazole (4.6 g, 21.1 mmol) was dissolved in dioxane (25 mL). HCl gas was bubbled through the solution for 4 h, then diethyl ether (50 mL) was added to give a suspension which was filtered off. The solid was dried in high vacuum to afford the title compound (3.03 g, 19.7 mmol, 93% yield). ¹H NMR (D₂O) δ: 7.00 (1H, d); m/z=119.0 [M+H]⁺.

### Example 2

### Preparation of 2-amino-5-fluorothiazole

5-Fluorothiazol-2-ylamino hydrochloride (5.50 g) was partitioned between Et₂O (100 mL) and saturated aqueous NaHCO₃ (100 mL). The aqueous phase was further extracted with Et₂O (100 mL), then the combined organic extracts were washed with brine (50 mL), before being dried (MgSO₄). Filtration and solvent evaporation furnished the free base (3.83 g).

### Example 3

### Preparation of (2R)-2-(4-cyclopropanesulfonylphenyl)-N-(5-fluorothiazol-2-yl)-3-(tetrahydropyran-4-yl)propionamide

A mixture of CH₂Cl₂ (1.35L) and DMF (35.91 mL, 0.465 mol, 1.5 eq) was cooled to −20° C. and oxalylchloride (39.4 mL, 0.465 mol, 1.5 eq) was added slowely. After stirring for 45 min (2R)-2-(4-cyclopropanesulfonylphenyl)-3-(tetrahydropyran-4-yl)propionic acid (Preparation 8, 105.0 g, 0.3101 mol, 1 eq) was added. The reaction was stirred at −20° C. for 1 h. Collidine (185 mL, 1.395 mol, 4.5 eq) was then slowly added and the reaction mixture was stirred for 15 min before the addition of 5-fluorothiazol-2-ylamine hydrochloride (Example 1b, 52.7 g, 0.341 mol, 1.1 eq) was at −15° C. The resulting suspension was kept at −15° C. for 2 h after which the ice bath was removed and the reaction slowly warmed up to RT over a period of 2 h. The mixture was evaporated to dryness to afford a semi-solid to which was added portionwise 4N HCl solution (1.5 mL). The product was extracted with ethylacetate (3L) and the organic fraction further washed with water (1L) and saturated NaHCO₃ solution (1L). The solvent was removed under partial vacuum to give the title compound (135 g). Characterising data was consistent with the formation of the title compound.

### Example 4

### Preparation of 2(R)-2-(4-cyclopropanesulfonylphenyl)-N-(5-fluorothiazol-2-yl)-3-((R)-3-oxocyclopentyl)propionamide

### a: (4-Cyclopropylsulfanylphenyl)oxoacetic acid

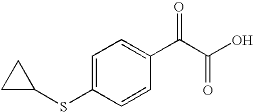

2M aqueous NaOH (163 mL) was added to a solution of ethyl (4-cyclopropylsulfanylphenyl)oxoacetate (40.62 g, 162.5 mmol) in EtOH (200 mL) and the stirred mixture heated at 60° C. for 2 h. After cooling, the mixture was concentrated to 150 mL and washed with ether (2×100 mL). Sufficient concentrated HCl was then added to adjust the pH to 1 and the resulting precipitate was extracted into EtOAc (2×300 mL). The combined organic phases were washed with water (3×100 mL), brine (200 mL) and dried (MgSO₄). Removal of the solvent gave the title compound: m/z (ES⁻)=221.0 [M−H⁺]⁻.

### b: (4-Cyclopropylsulfanylphenyl)acetic acid

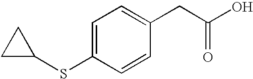

Hydrazine hydrate (14.19 g, 283.5 mmol) was cooled to −50° C. and (4-cyclopropylsulfanylphenyl)oxoacetic acid (12.6 g, 56.7 mmol) added in one portion. The vigorously-stirred slurry was warmed firstly to rt and then at 80° C. for 5 min. Solid KOH (8.76 g, 156.5 mmol) was added in four equal portions and the resulting solution heated at 100° C. for 20 h. On cooling to it, water (25 mL) was added and the aqueous phase washed with Et₂O (20 mL). The ethereal phase was itself washed with water (2×15 mL) and sufficient concentrated HCl added to the combined aqueous phases to adjust the pH to 1. The resulting precipitate was then extracted into EtOAc (2×300 mL) and the combined organic phases washed with water (3×100 mL), brine (200 mL) then dried (MgSO₄). Evaporation of the solvent gave the title compound: m/z (ES⁻)=207.1 [M−H⁺]⁻.

### e: 2-(4-Cyclopropylsulfanylphenyl)-N-(2(R)-hydroxy-1(R)-methyl-2-phenylethyl)-N-methylacetamide

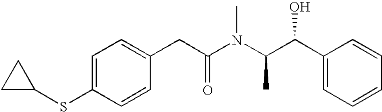

Anhydrous acetone (148 mL) was added to (4-cyclopropylsulfanylphenyl)-acetic acid (16.41 g, 78.8 mmol) and K₂CO₃ (32.67 g, 236.4 mmol) to form a slurry which was cooled to −10° C. with stirring. Neat trimethylacetyl chloride (10.2 mL, 82.74 mmol) was introduced dropwise, ensuring the temperature did not exceed −10° C. during the addition. The reaction mixture was stirred at −10° C. for 20 min, warmed to 0° C. for 20 min then cooled to −15° C. and solid (1(R),2(R))-(−)-pseudoephedrine (19.53 g, 118.2 mmol) was added in one portion. After 10 min, the reaction mixture was brought to rt, where stirring was continued for 1.5 h. Water (100 mL) was added and the mixture extracted with EtOAc (500 mL). The organic phase was washed with water (2×100 mL) and the combined aqueous layers back-extracted with EtOAc (2×250 mL). The combined organic layers were then washed with brine (100 mL) and dried (MgSO₄). The solvent was removed and the solid yellow residue recrystallized from EtOAc-IH to give the title compound: m/z (ES⁺)=356.1 [M+H]⁺.

### d: 2(R)-(4-Cyclopropylsulfanylphenyl)-3-(3(R)-oxocyclopentyl)propionic acid

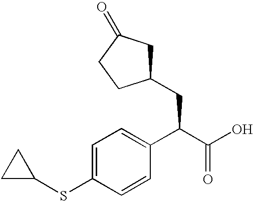

LHMDS (162 mL of a 1M solution in THF, 162 mmol) was diluted with anhydrous TMF (161 mL) and cooled to −20° C. with stirring. A solution of 2-(4-cyclopropylsulfanylphenyl)-N-(2(R)-hydroxy-1(R)-methyl-2-phenylethyl)-N-methylacetamide (30 g, 84.4 mmol) in anhydrous THF (245 mL) was added via cannula over 10 min, ensuring the reaction temperature remained below −15° C. throughout the addition. The reaction was allowed to warm to −7° C. over 30 min then cooled to −12° C. and a solution of 7(S)-iodomethyl-2(S),3(S)-diphenyl-1,4-dioxaspiro[4,4]nonane (27 g, 64.2 mmol) in a mixture of anhydrous THF (111 mL) and DMPU (18.9 mL) added via cannula over 10 min, ensuring the reaction temperature remained below −7° C. throughout. The reaction was warmed to 2° C. and stirred for 4.5 h before being poured into a mixture of toluene (770 mL) and 20% aqueous NH₄Cl (550 mL). After stirring vigorously, the organic layer was separated and washed with 20% aqueous NH₄Cl (550 mL) and brine (100 mL). The aqueous phases were combined and extracted with EtOAc (500 mL) which, after separation, was washed with brine (100 mL). The combined organic phases were dried (MgSO₄), filtered, evaporated and the resulting oil purified by flash chromatography (1H-EtOAc, 9:1 changing incrementally to 1:1) to give 2(R)-(4-cyclopropylsulfanylphenyl)-3-(2(S),3(S)-diphenyl-1,4-dioxaspiro[4.4]non-7(R)-yl)-N-(2(R)-hydroxy-1(R)-methyl-2-phenylethyl)-N-methylpropionamide: m/z (ES⁺)=648.3 [M+H]⁺. A stirred solution of this amide (30.7 g, 47.38 mmol) in 1,4-dioxane (62 mL) was diluted with 4.5M aqueous H₂SO₄ (61.5 mL) and the resulting mixture heated under gentle reflux for 18 h. After cooling on ice, water (162 mL) was added and the mixture extracted with EtOAc (250 mL). The aqueous layer was separated and extracted further with EtOAc (2×150 mL) and the combined organic phases washed with water (3×200 mL), ensuring the final wash was pH neutral, and brine (100 mL). After drying (MgSO₄) and filtering, the solvent was removed and the residue purified by flash chromatography (CH₂Cl₂ then CH₂Cl₂-THF, 5:1 changing to 3:1) to give the title compound: m/z (ES⁺)=305.1 [M+H]⁺.

### e: 2(R)-(4-Cyclopropanesulfonylphenyl)-3-(3(R)-oxocyclopentyl)propionic acid

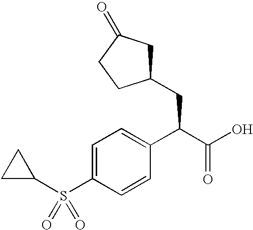

A stirred solution of 2(R)-(4-cyclopropylsulfanylphenyl)-3-(3(S)-oxocyclopentyl)propionic acid (5.0 g, 16.43 mmol) in CH₂Cl₂ (250 mL) was cooled to 1° C. on ice and 70% mCPBA (8.099 g, 32.85 mmol) added portionwise, maintaining the temperature below 3° C. After 6 h the solvent was removed and the residue purified by flash chromatography (1% AcOH in CH₂Cl₂ then THF) to give the title compound: m/z (ES⁺)=337.1 [M+H]⁺.

### f: 2 (R)-2-(4-Cyclopropanesulfonylphenyl)-N-(5-fluorothiazol-2-yl)-3-((R)-3-oxocyclopentyl)propionamide

A solution of 2(R)-(4-cyclopropanesulfonylphenyl)-3-(3(R)-oxocyclopentyl)propionic acid (893 mg, 2.65 mmol) in anhydrous CH₂Cl₂ (38 mL) was cooled to 0° C. and a solution of oxalyl chloride (0.408 g, 3.21 mmol) in anhydrous CH₂Cl₂ (2 mL) added-dropwise, maintaining the temperature at 0° C. during the addition. Dry DMF (0.08 mL) was added and the reaction mixture stirred 2.5 h. A solution of 2-amino-5-fluorothiazole free base (Example 2, 345 mg, 2.92 mmol) in anhydrous CH₂Cl₂ (6 mL) was introduced slowly, followed by pyridine (0.53 mL, 5.31 mmol) and the mixture stirred at 0° C. for 2 h then at rt overnight. The solution was diluted with CH₂Cl₂ (150 mL) and washed with aqueous 5% w/v citric acid (2×30 mL), saturated aqueous NaHCO₃ (2×30 mL), water (50 mL) and brine (50 mL). The organic phase was dried (MgSO₄), evaporated and the residue purified by flash chromatography (IH-EtOAc, 3:2) to afford the title compound. Characterising data was consistent with the formation of the title compound.

### Example 5

### a) 2-Acetamido-5-fluorothiazole

2-Acetamidothiazole (215 mg, 1.51 mmol) was added to a stirred solution of Selectfluor® (714 mg, 2.02 mmol) in anhydrous MeCN (20 mL). The mixture was heated under reflux for 16.5 h, then the solvent was evaporated off under reduced pressure. The residue was partitioned between EtOAc (60 mL) and H₂O (30 mL). The aqueous phase was extracted further with EtOAc (30 mL), then the combined organic extracts were washed with H₂O (30 mL) and saturated aqueous NaHCO₃ (30 mL), before being dried (MgSO₄). Filtration, solvent evaporation, and flash chromatography (Isohexane-EtOAc, 4:1 to 1:1) furnished the title compound as a white solid (117 ng, 48%): RT=2.40 min; 111/Z=161.0 [M+H]⁺.

### b) 5-Fluorothiazol-2-ylamine hydrochloride

A stirred mixture of 2-acetamido-5-fluorothiazole (6.3 g, 39.4 mmol) and 2M HCl (150 mL) was warmed at 70-75° C. for 16 h. The reaction was evaporated to dryness, then PhMe was added, before being evaporated off to remove any residual water. The remainder was stirred with THF (50 mL), before being collected and dried to furnish the title compound: δ_{H} (D₂O): 7.00 (1H, d), m/z=119.0 [M+H]⁺.

1. A process for the production of a compound of formula (I): 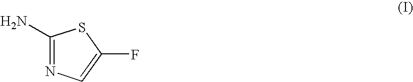
or an acid addition salt thereof, comprising fluorination of a compound of formula (II): 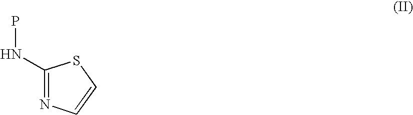
wherein P is a protecting group followed by removal of the protecting group and optional salt formation.

2. The process according to claim 1 wherein the protecting group is acetyl, pivaloyl, or tert-butoxycarbonyl.

3. The process according to claim 1 wherein the protecting group is tert-butoxycarbonyl.

4. The process according to claim 1 wherein the fluorination reagent is an electrophilic fluorinating agent.

5. The process according to claim 4 wherein the fluorination reagent comprises an active N-fluorine bond.

6. The process according to claim 5 wherein the fluorination reagent is a N-fluorosulfonimide.

7. The process according to claim 6 wherein the fluorination reagent is N-fluorobenzenesulfonimide.

8. The process according to claim 1 wherein the compound of formula (II) is deprotonated using an organolithium reagent.

9. The process according to claim 8 wherein the compound of formula (II) is deprotonated using about 2 equivalents of tert-butyl lithium.

10. The process according to claim 1 which is conducted in a polar aprotic solvent.

11. The process according to claim 8 wherein the solvent is tetrahydrofuran.

12. The process according to claim 1 wherein the fluorination reagent is 1-chloromethyl-4-fluoro-1,4-diazoniabicyclo[2.2.2]octane bis(tetrafluoroborate).

13. The process according to claim 1 wherein the salt of the compound of formula (I) is the hydrochloride salt.

14. A process for the production of a compound of formula (III), or a pharmaceutically acceptable salt thereof: 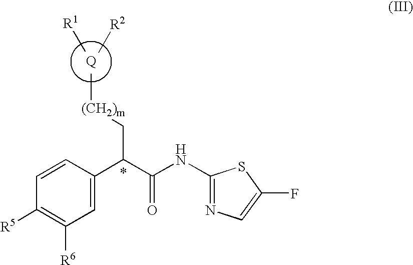
or a pharmaceutically acceptable salt thereof, wherein:
Q is an aryl, a 5- or 6-membered heteroaryl, or a 4-8-membered hetrocyclic ring;
R¹ and R² each independently are hydrogen, hydroxy, halogen, cyano, nitro, vinyl, ethynyl, methoxy, OCFₙH₃₋ₙ—N(C₀₋₄alkyl)C₀₋₄alkyl), CHO, or C₁₋₂alkyl optionally substituted with 1-5 substituents independently selected from: halogen, hydroxy, cyano, methoxy, —N(C₀₋₂alkyl)(C₀₋₂alkyl), SOCH₃, and SO₂CH₃ substituents; or R¹ and R² together form a carbocyclic or heterocyclic ring; or R¹ and R² may be taken together to represent an oxygen atom attached to the ring via a double bond;
R⁵ and R⁶ each independently are hydrogen, hydroxy, halogen, cyano, nitro, CO₂R⁷, CHO, COR⁸, C(OH)R⁷R⁸, C(═NOR⁷)R⁸, CONR⁹R¹⁰, SR⁷, SOR⁸, SO₂R⁸, SO₂NR⁹R¹⁰, CH₂NR⁹R¹⁰, NR⁹R¹⁰, N(C₀₋₄alkyl)SO₂R⁸, NHCOR⁷, or C₁₋₄alkyl group, C₂₋₄alkenyl group, C₂₋₄alkynyl group, C₁₋₄alkoxy group, aryl group, or heteroaryl group, wherein any group optionally is substituted with 1-6 substituents independently selected from: halogen, cyano, nitro, hydroxy, C₁₋₂alkoxy, —N(C₀₋₂alkyl)(C₀₋₂alkyl), C₁₋₂alkyl, CFₙH₃₋ₙ, aryl, heteroaryl, —COC₁₋₂alkyl, CON(C₀₋₂alkyl)C₀₋₂alkyl), SCH₃, SOCH₃, SO₂CH₃, or —SO₂N(C₀₋₂alkyl)(C₀₋₂alkyl) substituents, and R⁵ and R⁶ together form a 5-8-membered carbocyclic or hetrocyclic ring:
R⁷ is hydrogen, or C₁₋₄alkyl group, C₂₋₄alkenyl group, C₂₋₄alkynyl group, C₃₋₇cycloalkyl group, aryl group, heteroaryl group, or 4-7-membered heterocyclic group, wherein any group optionally is substituted with 1-6 substituents independently selected from: halogen, cyano, nitro, hydroxy, C₁₋₂alkoxy, —N(C₀₋₂alkyl)(C₀₋₂alkyl), C₁₋₂alkyl, C₃₋₇cycloalkyl, 4-7-membered hetrocyclic ring, CFₙH₃₋ₙ, aryl, heteroaryl, CO₂H, —COC₁₋₂alkyl, —CON(C₀₋₂alkyl), (C₀₋₂alkyl), SOCH₃, SO₂CH₃, and —SO₂N(C₀₋₂alkyl)(C₀₋₂alkyl) substituents;
R⁸ is C₁₋₄aklyl group, C₂₋₄alkenyl group, C₂₋₄alkynyl group, C₃₋₇cycloalkyl group, aryl group, heteroaryl group, or 4-7-membered heterocyclic group, wherein any group optionally is substituted with 1-6 substituents independently selected from: halogen, cyano, nitro, hydroxy, C₁₋₂alkoxy, —N(C₀₋₂alkyl)(C₀₋₂alkyl), C₁₋₂alkyl, C₃₋₇cycloalkyl, 4-7-membered heterocyclic ring, CFₙH₃₋ₙ, aryl, heteroaryl, CO₂H, COC₁₋₂alkyl, —CON(C₀₋₂alkyl(C₀₋₂alkyl), SOCH₃, SO₂CH₃, and —SO₂N(C₀₋₂alkyl)(C₀₋₂alkyl) substituents;
R⁹ and R¹⁰ each independently are hydrogen, or C₁₋₄alkyl group, C₃₋₇cycloalkyl group, aryl group, heteroaryl group, or 4-7-membered heterocyclic group, wherein any group optionally is substituted with 1-6 substituents independently selected from: halogen, cyano, nitro, hydroxy, C₁₋₂alkoxy, —N(C₀₋₂alkyl)(C₀₋₂alkyl), C₁₋₂alkyl, C₃₋₇cycloalkyl, 4-7-membered heterocyclic ring, CFₙH₃₋ₙ, aryl, heteroaryl, COC₁₋₂alkyl, —CON(C₀₋₂alkyl), (C₀₋₂alkyl), SOCH₃, SO₂CH₃, and —SO₂N(C₀₋₂alkyl)(C₀₋₂alkyl) substituents; or R⁹ and R¹⁰ together form a 6-8-membered heterobicyclic ring system or a 4-8-membered heterocylic ring which optionally is substituted with 1-2 independent C₁₋₂alkyl, CH₂OCH₃, COC₀₋₂alkyl, hydroxy, or SO₂CH₃ substituents;
n is 1, 2 or 3; and
m is 0 or 1;
which comprises the condensation of a compound of formula (I) produced according to claim 1 or a salt thereof, with a carboxylic acid of formula (IV) or an activated derivative thereof: 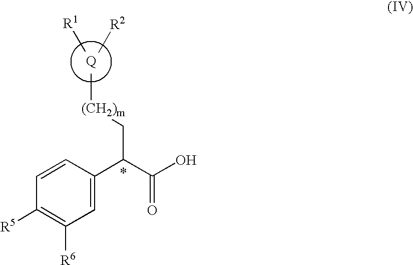
wherein R¹, R², R⁵, R⁶, Q and m are as defined above.

15. The process according to claim 14 wherein in the compounds of formula (III) the carbon atom linking the aryl ring and Q-bearing sidechain to the carbonyl carbon is in the (R)-configuration.

16. The process according to claim 14 wherein in the compounds of formula (III):
Q is 4-tetrahydropyranyl;
R¹ and R² are hydrogen;
R⁵ is SO₂R⁸, or SO₂NR⁹R¹⁰;
R⁶ is hydrogen;
R⁸ is a C₃₋₅cycloalkyl group or a 4-6-membered heterocyclic group, and, in addition;
R⁹ and R¹⁰ are independently C₀₋₄alkyl, provided that R⁹ and R¹⁰ are not both hydrogen; and
m is 0.

17. The process according to claim 14 wherein in the compounds of formula (III) R⁵ is SO₂cyclopropyl.

18. A process for the production of a compound of formula (VII), or a pharmaceutically acceptable salt thereof: 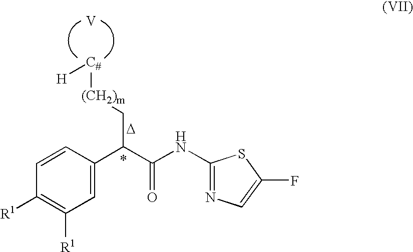
wherein V is (CH₂)ₖ where one CH₂ group may optionally be replaced by CH(OH), C═O, C═NOH, C═NOCH₃, CHX, CXX¹, CH(OCH₃) CH(OCOCH₃), CH(C₁₋₄alkyl), or X and X¹ are independently selected from fluoro and chloro;
R¹ and R² re independently selected from hydrogen, halogen, hydroxy, amino, cyano, nitro, SR³, SOR³, SO₂R³, SO₂NR⁴R⁵, NHSO₂R³, or a C₁₋₄alkyl, C₂₋₄alkenyl, C₂₋₄alkynyl, C₁₋₄alkoxy, or heteroaryl group, wherein any group is optionally substituted with 1 to 5 substituents independently selected from halogen, cyano, nitro, hydroxy, C₁₋₂alkoxy, —N(C₀₋₂alkyl), C₁₋₂alkyl, CFₙH₃₋ₙ, aryl, heteroaryl, —CON(C₀₋₂alkyl)(C₀₋₂alkyl), SCH₃, SOCH₃, SO₂CH₃, and —SO₂N(C₀₋₂alkyl)(C₀₋₂alkyl);
R³ is a group, C₃₋₇cycloalkyl group, aryl group, heteroaryl group, or 4- to 7-membered heterocyclic group, wherein any group, any group is optionally substituted with 1 to 5 substituents independently selected from halogen, cyano, nitro, hydroxy, C₁₋₂alkoxy, —N(C₀₋₂alkyl)(C₀₋₂alkyl), C₁₋₂alkyl, C₃₋₇cycloalkyl, 4- to 7-membered heterocyclic ring, CFₙH₃₋ₙ aryl, heteroaryl, COC₁₋₂alkyl, —CON(C₀₋₂alkyl)(C₀₋₂alkyl), SOCH₃, SO₂CH₃, and —SO₂N(C₀₋₂alkyl)(C₀₋₂alkyl);
R⁴ and R⁵ are independently hydrogen, or a C₁₋₄alkyl group, C₃₋₇cycloalkyl group, aryl group, heteroaryl group, or 4- to 7-membered heterocyclic group, wherein any group is optionally substituted with 1 to 5 substituents independently selected from halogen, cyano, nitro, hydroxy, C₁₋₂alkoxy, —N(C₀₋₂alkyl)(C₀₋₂alkyl), C₁₋₂alkyl, C₃₋₇cycloalkyl, 4- to 7-membered heterocyclic ring, CFₙH₃₋ₙ, aryl, heteroaryl, —CON(C₀₋₂alkyl, C₀₋₂alkyl, SOCH₃, SO₂CH₃, and —SO₂N(C₀₋₂alkyl)(C₀₋₂alkyl); or R⁴ and R⁵ together form a 4- to 8-membered heterocyclic ring which is optionally substituted with 1 or 2 substituents independently selected from C₁₋₂alkyl and hydroxy;
k is an integer form 2 to 7;
m is 0 or 1; and
n is 1, 2 or 3
which comprises the condensation of a compound of formula (I) produced according to claim 1 or a salt thereof, with a carboxylic acid of formula (VIII) or an activated derivative thereof: 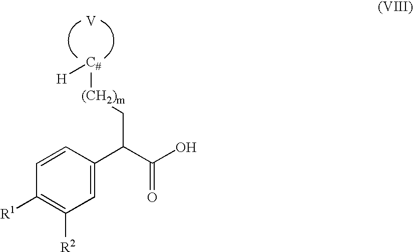
wherein V, R¹, R² and m are as defined for formula (VII).

19. The process according to claim 18 wherein in the compounds of formula (VII) the group formed by 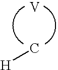
represents oxocycloalkyl or hydroxycycloalkyl.

20. The process according to claim 18 wherein in the compounds of formula (VII) R¹ and R² are not both hydrogen.

21. The process according to claim 20 wherein in the compounds of formula (VII) R¹ is SO₂C₃₋₄cycloalkyl.

22. The process according to claim 18 wherein in the compound of formula (VII) R⁴ and R⁵ are independently hydrogen or C₁₋₄alkyl.

23. The process according to claim 18 wherein in the compounds of formula (VII) in is 0.

24. The process according to claim 18 wherein in the compounds of formula (VII) k is 4 or 5.

25-28. (canceled)
